(19) United States
(12) Patent Application Publication
(10) Pub. No.: US 2023/0151114 A1
(43) Pub. Date: May 18, 2023

(54) COMPOSITIONS AND METHODS OF TREATMENT OF SICKLE CELL ANEMIA AND BETA-THALASSEMIA

(71) Applicant: Cleveland State University, Cleveland, OH (US)

(72) Inventors: Merlin Nithya Gnanapragasam, Solon, OH (US); Mahesh Ramamoorthy, Solon, OH (US)

(73) Assignee: Cleveland State University, Cleveland, OH (US)

(21) Appl. No.: 17/987,058

(22) Filed: Nov. 15, 2022

Related U.S. Application Data
(60) Provisional application No. 63/279,232, filed on Nov. 15, 2021.

(51) Int. Cl. C07K 16/30; A61K 31/7088

(52) U.S. Cl. CPC C07K 16/3092; A61K 31/7088

(57) ABSTRACT

Therapeutic compositions and methods for treating a patient having sickle cell anemia or beta-thalassmia by administering, in therapeutically effective amounts, a composition of an effective amount of an inhibitor of Pumilio-1.

The present application claims the priority benefit of U.S. Provisional Pat. Application Serial No. 63/279,232, Nov. 15, 2021, the disclosure of which is incorporated herein by reference.

### BACKGROUND

The present disclosure relates to compositions, treatments, and methods for treating a patient by administering, in therapeutically effective amounts, a composition for the treatment of sickle cell anemia and beta-thalassemia.

Normal adult hemoglobin comprises four globin proteins, two of which are alpha (α) proteins and two of which are beta (β) proteins. During mammalian fetal development, particularly in humans, the fetus produces fetal hemoglobin, which comprises two gamma (γ)-globin proteins instead of the two β-globin proteins. At some point during fetal development or infancy, depending on the particular species and individual, a globin switch occurs, referred to as the “fetal switch”, at which point, erythrocytes in the fetus switch from making predominantly γ-globin to making predominantly β-globin. The developmental switch from production of predominantly fetal hemoglobin or HbF (α2γ2) to production of adult hemoglobin or HbA (α2β2) begins at about 28 to 34 weeks of gestation and continues shortly after birth until HbA becomes predominant. This switch has been thought to result primarily from decreased transcription of the gamma-globin genes and increased transcription of beta-globin genes. On average, the blood of a normal adult contains only about 1% HbF, though residual HbF levels have a variance of over 20 fold in healthy adults (Atweh, Semin. Hematol. 38(4):367-73 (2001); Oilman JG, et al., Br. J. Haematol. 1988;68(4):455-458)).

Hemoglobinopathies encompass a number of anemias of genetic origin in which there is a decreased production and/or increased destruction (hemolysis) of red blood cells (RBCs). These also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickled) hemoglobin (HbS). HbS RBCs are more fragile than normal RBCs and undergo hemolysis more readily, leading eventually to anemia (Atweh, Semin. Hematol. 38(4):367-73 (2001)).

Recently, the search for treatment aimed at reduction of globin chain imbalance in patients with β-hemoglobinopathies has focused on the pharmacologic manipulation of fetal hemoglobin (α2γ2; HbF). The therapeutic potential of such approaches is suggested by observations of the mild phenotype of individuals with co-inheritance of both homozygous β-thalassemia and hereditary persistence of fetal hemoglobin (HPFH), as well as by those patients with homozygous β°-thalassemia who synthesize no adult hemoglobin, but in whom a reduced requirement for transfusions is observed in the presence of increased concentrations of fetal hemoglobin. Furthermore, it has been observed that certain populations of adult patients with β chain abnormalities have higher than normal levels of fetal hemoglobin (HbF), and have been observed to have a milder clinical course of disease than patients with normal adult levels of HbF. For example, a group of Saudi Arabian sickle-cell anemia patients who express 20-30% HbF have only mild clinical manifestations of the disease (Pembrey, et al., Br. J. Haematol. 40: 415-429 (1978)). It is now accepted that hemoglobin disorders, such as sickle cell anemia and the β -thalassemias, are ameliorated by increased HbF production. (Reviewed in Jane and Cunningham Br. J. Haematol. 102: 415-422 (1998) and Bunn, N. Engl. J. Med. 328: 129-131 (1993)).

As mentioned earlier, the switch from fetal hemoglobin to adult hemoglobin (α2γ2; HbA) usually proceeds within six months after parturition. However, in the majority of patients with β-hemoglobinopathies, the upstream γ globin genes are intact and fully functional, so that if these genes become reactivated, functional hemoglobin synthesis could be maintained during adulthood, and thus ameliorate disease severity (Atweh, Semin. Hematol. 38(4):367-73 (2001)). Unfortunately, the in vivo molecular mechanisms underlying the globin switch are not well understood.

Overall, identification of molecules that play a role in the globin switch is important for the development of novel therapeutic strategies that interfere with adult hemoglobin and induce fetal hemoglobin synthesis. Such molecules would provide new targets for the development of therapeutic interventions for a variety of hemoglobinopathies in which reactivation of fetal hemoglobin synthesis would significantly ameliorate disease severity and morbidity.

### BRIEF DESCRIPTION

The present disclosure is directed to compositions and methods for treating sickle cell anemia or beta-thalassmia. Also disclosed are compositions and methods for increasing fetal hemoglobin levels.

Disclosed, in some embodiments, is a method of treating sickle cell anemia or beta-thalassmia, the method including: administering to a patient a composition of an effective amount of an inhibitor of Pumilio-1 (PUM1).

The inhibitor may be an antibody or fragment thereof, a nucleic acid, or a small molecule.

Disclosed, in other embodiments, is a pharmaceutical composition containing an inhibitor of Pumilio-1 and a pharmaceutically acceptable carrier.

Disclosed, in further embodiments, is a method for increasing fetal hemoglobin levels, the method including: administering an effective amount of the pharmaceutical composition, whereby fetal hemoglobin expression is increased relative to the amount prior to administration of the composition comprising an inhibitor of Pumilio-1.

Disclosed, in additional embodiments, is a method for increasing fetal hemoglobin levels, the method including: genome editing, whereby fetal hemoglobin expression is increased relative to the amount prior to the genome editing; and/or utilizing antisense oligonucleotides such as siRNA, whereby fetal hemoglobin expression is increased relative to the amount prior to the use of antisense oligonucleotides; and/or utilizing RNA decoy technology, whereby fetal hemoglobin expression is increased relative to the amount prior to the use of the RNA decoy technology.

In some embodiments, the genome editing decreases PUM1.

The use of antisense oligonucleotides may decrease PUM1.

In some embodiments, the use of RNA decoy technology decreases PUM1.

The fetal hemoglobin level may be at least 5% higher in populations treated, than a comparable, control population, wherein no treatment occurs.

These and other non-limiting aspects of the disclosure are more particularly set forth below.

### BRIEF DESCRIPTION OF THE DRAWINGS

FIG. 1 illustrates RNA-Seq analysis on Eklf+/+ and -/- murine erythroid cells.

FIG. 2 illustrates PUM1 in cytoplasm before and after erythroid differentiation.

FIG. 3 illustrates the effect of PUM1 on globins.

FIG. 4 illustrates a modest increase in γ-globin transcript levels.

FIG. 5 illustrates γ-globin protein levels increase during erythroid differentiation.

FIG. 6 illustrates the effects of PUM1 levels.

FIG. 7 illustrates that PUM1 affects the translation of HBG1.

FIG. 8 illustrates that knockdown of PUM1 did not affect erythropoiesis in either HUDEP2 or primary human erythroid cells.

FIG. 9 illustrates that knockdown of PUM1 did not lead to changes in the levels of certain proteins.

FIG. 10 illustrates a mutation and a comparison of a patient with a healthy parent.

FIG. 11 is the complete blood count of a patient suggesting that elevated fetal hemoglobin was not due to anemia.

FIG. 12 includes western blots for CRISPR-Cas9 based PUM1 knockout in human erythroid cells.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named components/steps and allowing the presence of other components/steps. The term “comprising” should be construed to include the term “consisting of”, which allows the presence of only the named components/steps.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of an effective amount of a compound that reduces the symptoms of a disease or disorder. The compound may be comprised in a pharmaceutically acceptable carrier. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington’s Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated. Upon formulation, solutions may be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

As used herein, the term “small molecule” refers to a chemical agent including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

A “nucleic acid”, as described herein, can be RNA or DNA, and can be single or double stranded, and can be selected, for example, from a group including: nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, molecules, ribozymes, small inhibitory nucleic acid sequences, for example but are not limited to RNAi, shRNAi, siRNA, micro RNAi (mRNAi), antisense oligonucleotides etc.

In connection with “increasing fetal hemoglobin levels” indicates that fetal hemoglobin is at least 5% higher in populations treated with a PUM1 inhibitor, than a comparable, control population, wherein no PUM1 inhibitor is present. It is preferred that the fetal hemoglobin expression in a PUM1 inhibitor treated patient is at least 5% higher, at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than a comparable control treated patient.

As disclosed herein, it is an object of the present invention to provide a method for increasing fetal hemoglobin levels in a mammal.

Pumilio 1 or PUM1 has the sequence for which can be found at GenBank Accession Nos. NM—001020658.1 and NP—001018494.1.

Effective reversion of expression from adult β-globin to its fetal form, γ-globin, in adult erythrocytes, can ameliorate debilitating diseases such as sickle cell anemia and β-thalassemia. Pumilo-1 (PUM1), an RNA binding protein, is a direct post-transcriptional regulator of switching from the fetal to adult form of globin. PUM1 expression, regulated by the erythroid master transcription factor, Erythroid Kruppel-like factor (EKLF/KLF1), peaks during erythroid differentiation, binds γ-globin mRNA, impacts γ-globin mRNA stability and translation, and culminates in reduced γ-globin protein levels. Suppression of PUM1 increases γ-globin protein levels without affecting β-globin expression in human erythroid cells. Importantly, targeting PUM1 does not limit erythropoiesis progression, providing a potentially safe and effective treatment strategy in sickle cell anemia and β-thalassemia. In support of this idea, we report higher fetal hemoglobin (HbF) levels in a patient with a novel PUM1 mutation in the RNA binding domain, suggesting that PUM1 mediated post-transcriptional regulation of γ-globin is a critical step during human globin switching.

Erythroid differentiation involves a series of steps orchestrated by the definitive erythroid master regulator, EKLF/KLF1. We had previously created an ex vivo primary cell system to expand our understanding of how EKLF mediates the precise changes leading to terminal erythropoiesis and enucleation.

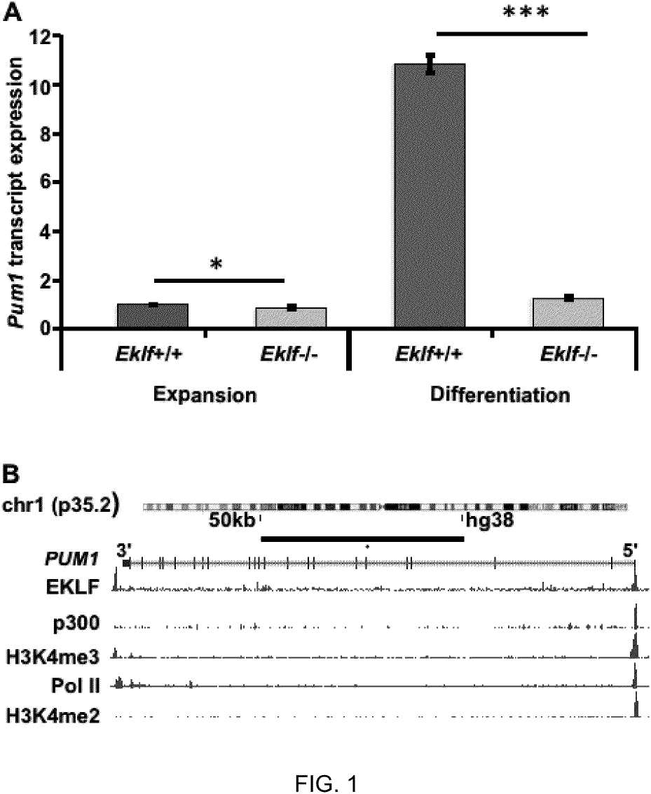

As shown in FIGS. 1A and 1B, RNA-Seq analysis on the Eklf+/+ and -/- murine erythroid cells, along with ChlP-Seq analyses in human erythroid cells, identified a novel EKLF target, an RNA binding protein Pumilio1 (PUM1), which is upregulated specifically during erythroid terminal differentiation. PUM1 is a member of the PUF family of sequence-specific RNA-binding proteins and acts as a post-transcriptional repressor by binding to the 3′-UTR of mRNA targets and impairing their stability and/or translational efficiency.

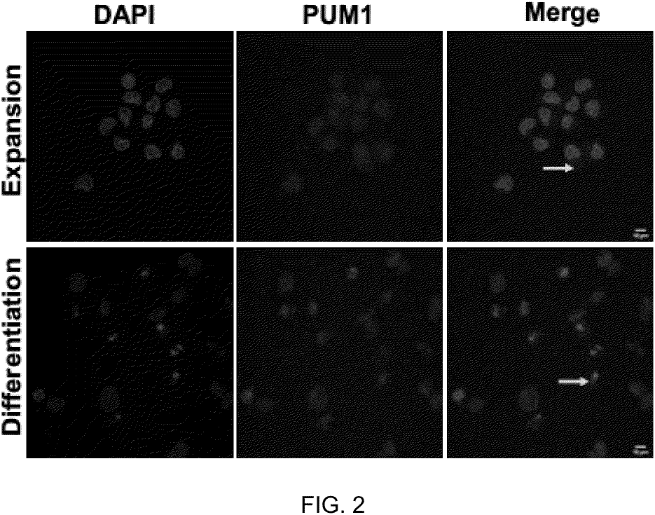

As shown in FIG. 2, consistent with its function, we observe PUM1 in the cytoplasm before and after erythroid differentiation. PUM1 plays an important role in early embryonic development and in the maintenance of hematopoietic stem cells, while mutations in PUM1 contribute to neurological diseases and cancer. However, to date its role in erythropoiesis is not known.

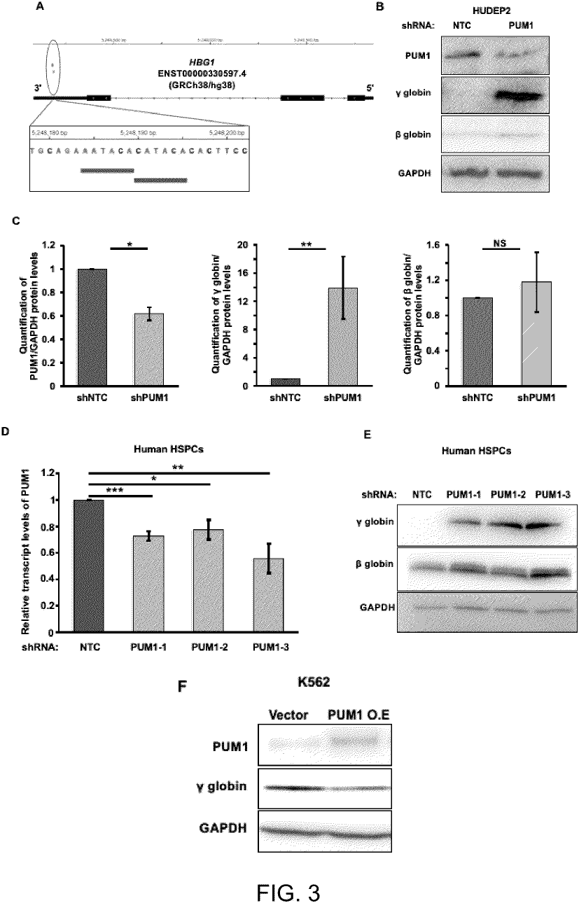

Since EKLF is a known regulator of γ-globin to β-globin switching and since PUM1 is under the control of EKLF, in order to identify one of the potential functions of PUM1 in erythroid differentiation, as shown in FIG. 3A, we first tested its ability to bind to erythroid specific mRNAs, specifically γ-globin and β-globin. γ-globin is expressed from two genes, HBG1 and HBG2, both arising as an ancestral duplication event. We noted that the ^{A}γ (HBG1) gene but not the ^{G}γ (HBG2) form of the fetal globin (γ-globin) has two core PUM1 consensus binding sites in its 3′-UTR, while none of the other globins, fetal or adult, share this feature.

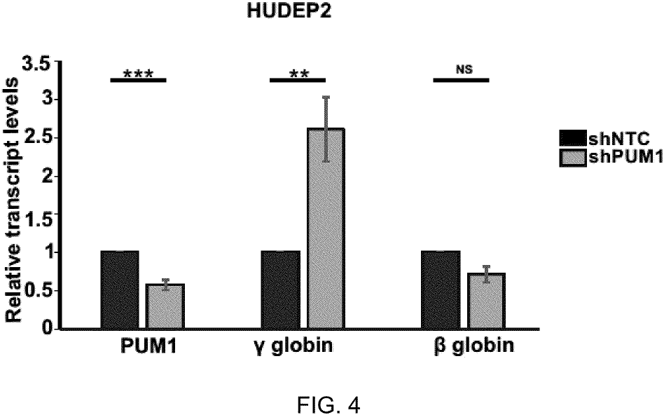

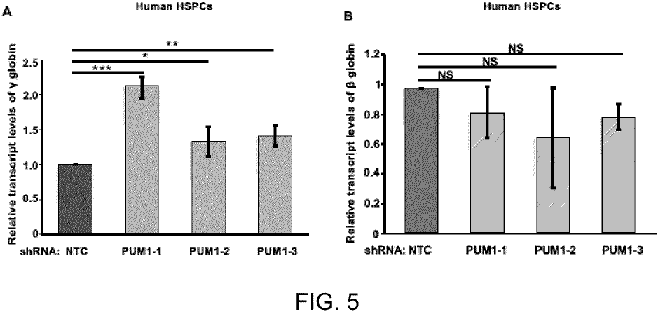

Since, γ-globin comprises less than 1% of all globins in the adult blood cell, we hypothesized that PUM1 could suppress γ-globin expression specifically in the adult red blood cells. To test our hypothesis, we knocked down PUM1 in HUDEP2, an immortalized human erythroid progenitor cell line, and examined γ-globin levels. While we observed a modest increase (-2.5-fold) in the γ-globin transcript levels (FIG. 4) the increase in the γ-globin protein levels after PUM1 knockdown was more dramatic, more than 12-fold (FIGS. 3B and 3C). Similar results are also observed in primary erythroid cells derived from CD34+ human Hematopoietic Stem and Progenitor Cells (HSPC), with a modest PUM1 knockdown leading to a robust increase in γ-globin protein levels during erythroid differentiation (FIGS. 3D and 3E; and FIGS. 5A and 5B). Conversely, overexpression of PUM1 in K562 erythroleukemia cells that express high endogenous levels of fetal hemoglobin showed reduction of γ-globin (FIG. 3F).

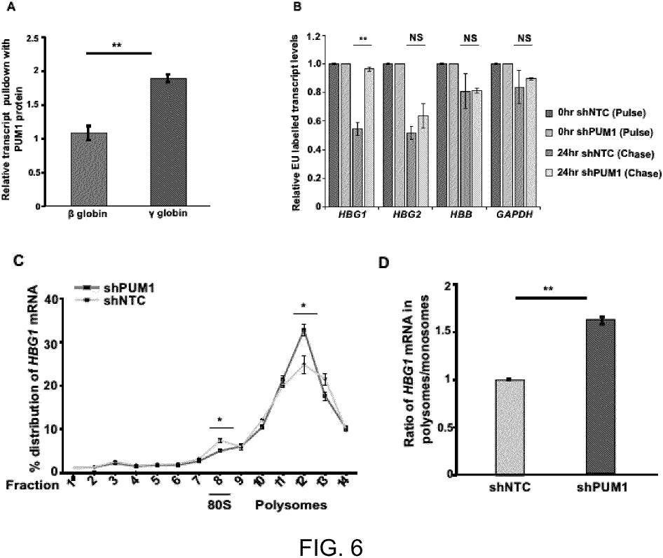

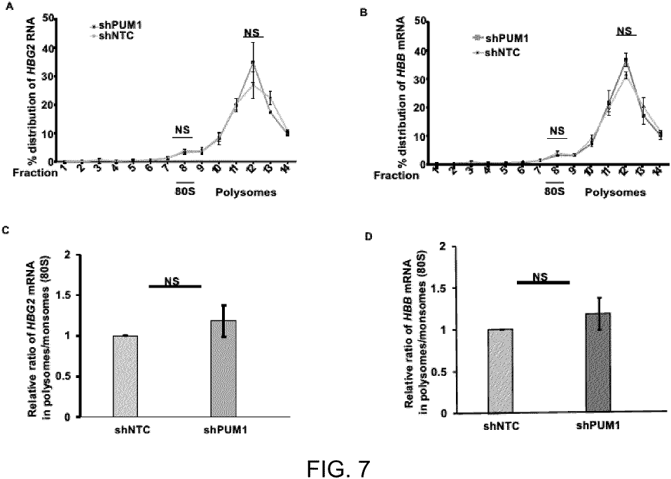

To address the effects of PUM1 on mRNA and protein levels, we next tested the role of PUM1 in γ-globin mRNA stability and protein translation. PUM1’s role in regulating gene expression in mammals has been in particular attributed to target mRNA degradation by associating with the Ccr-Not complex and/or translation inhibition of target mRNAs by disrupting the activity of poly(A)-binding protein. First, we tested if PUM1 mediates γ-globin levels by directly binding to γ-globin mRNA. As shown in FIG. 6A, RNA immunoprecipitation (RIP) of PUM1 pulled down γ-globin mRNA in comparison to β-globin mRNA. These results indicate that PUM1’s role in γ-globin regulation may be unique and direct. We then performed a nascent mRNA degradation assay where we pulsed RNA with ethylene uridine (EU) ribonucleotide homologs and after washing it off, analyzed the newly synthesized EU-incorporated mRNA at different time points. We observed that while the EU incorporated HBG1 and HBG2 mRNA levels were reduced over time in the control cells, HBG1 mRNA levels were relatively stabilized when PUM1 is knocked down, suggesting that PUM1 plays an erythroid specific role in the degradation of HBG1 mRNA (FIG. 6B). Next, we performed polysome profiling of the control and PUM1 knocked down HUDEP2 cells and observed a specific increase in the HBG1 mRNA levels in polyribosomal fractions as compared to the monosomal fraction(s), suggesting that HBG1 mRNA is more actively translated under the conditions of reduced PUM1 levels (FIGS. 6C and 6D). This was not the case with the HBG2 mRNA or the β-globin mRNA, affirming that PUM1 affects the translation of HBG1 (FIGS. 7A-7D). These results demonstrate that PUM1 regulates HBG1 both at the level of mRNA stability and translation. PUM1 expression increases during erythroid differentiation indicating that PUM1 serves as a post-transcriptional regulator of γ-globin in adult human erythroid cells.

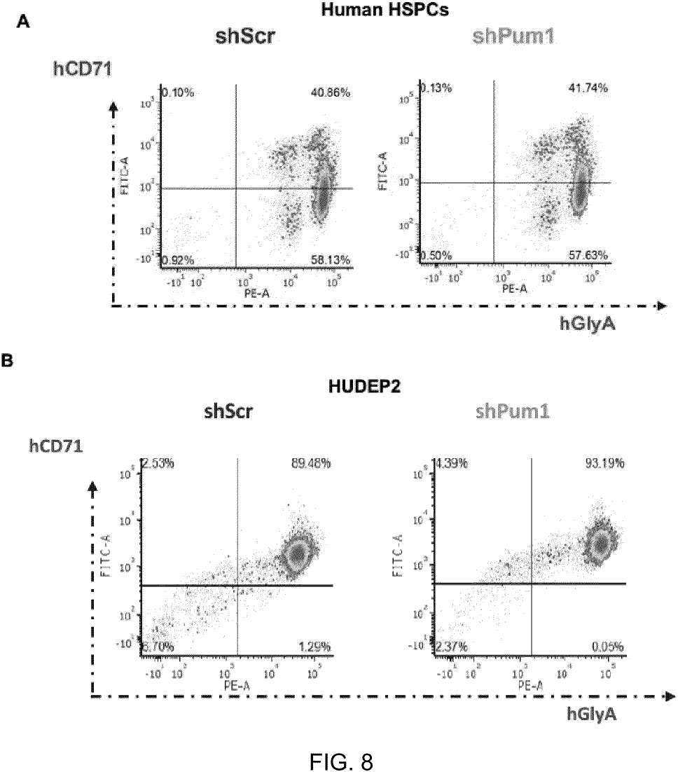

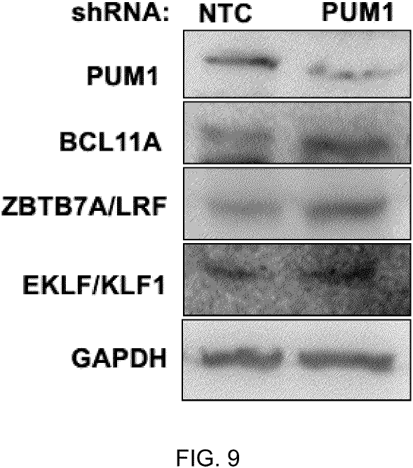

Our data also underline the importance of the fine-tuned homeostasis required for PUM1 protein levels, as we observed that even slight perturbations in PUM1 levels result in gross γ-globin changes, similar to what was previously reported in patient mutations that reduced PUM1 levels by 25% in other tissues (see Gennarino, V. A. et al. A Mild PUM1 Mutation Is Associated with Adult-Onset Ataxia, whereas Haploinsufficiency Causes Developmental Delay and Seizures. Cell 172, 924-936.e11 (2018)). Importantly, knockdown of PUM1 did not affect erythropoiesis in either HUDEP2 or in primary human erythroid cells (FIG. 8). We also investigated if PUM1 regulates known γ-globin regulators such as KLF1, BCL11A, and ZBTB7A. As shown in FIG. 9, knockdown of PUM1 did not lead to the changes in the levels of these proteins.

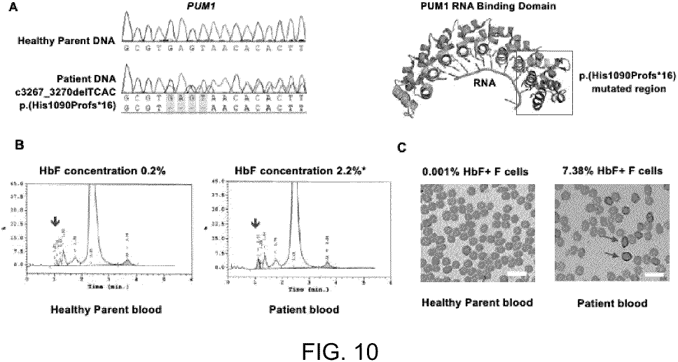

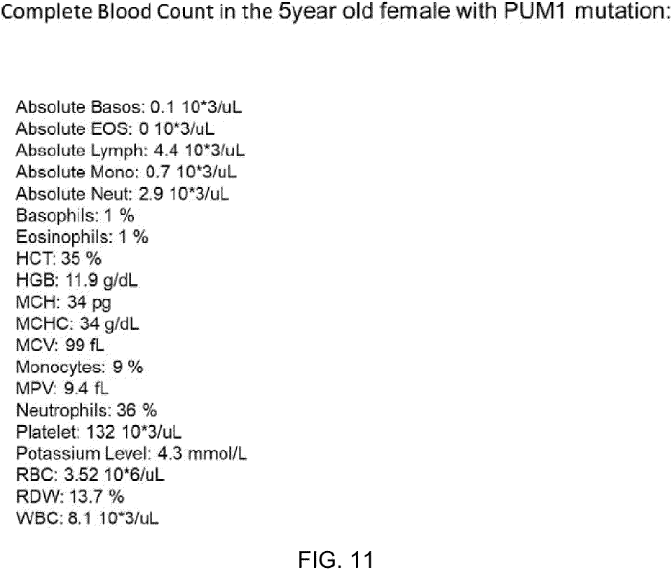

PUM1’s diverse roles in human pathology implies that it has distinct cell-type dependent roles during development. Therefore, we further decided to investigate if patient mutations in PUM1 could result in high fetal hemoglobin levels in the blood. We identified a 5 yr old child with PADDAS (PUM1-associated developmental disability, ataxia, and seizure) harboring a novel heterozygous PUM1 mutation (p.(His1090Profs*16); c.3267_3270deITCAC). The mutation, a frameshift in the RNA binding domain, introduces 16 new amino acids and a premature stop codon (FIG. 10A). We analyzed the blood of the patient and compared the results with the blood from a healthy parent. We observed elevated fetal hemoglobin levels in the patient (over the accepted reference range), with more than 10-fold increase over the healthy parent’s level, as analyzed by HPLC (FIG. 10B) and by Modified Kleihauer-Betke staining for F cells (FIG. 10C). The complete blood count in the patient suggests that elevated fetal hemoglobin was not due to anemia (FIG. 11).

While transcriptional and epigenetic regulation of β-globin switching and its therapeutic relevance has been studied extensively, post-transcriptional regulation of β-globin genes is poorly understood. A handful of studies point to the physiological and clinical relevance for this regulation (see Lumelsky, N. L. & Forget, B. G. Negative regulation of globin gene expression during megakaryocytic differentiation of a human erythroleukemic cell line. Mol. Cell. Biol. 11, 3528-3536 (1991) and Chakalova, L. et al. The Corfu δβ thalassemia deletion disrupts γ-globin gene silencing and reveals post-transcriptional regulation of HbF expression. Blood 105, 2154-2160 (2005)). Further, chemicals such as butyrate and salubrinal have also been demonstrated to increase γ-globin levels independent of its transcriptional activation. However, the mechanisms underlying the post-transcriptional regulation of γ-globin have thus far remained unclear. Here, we report the identification of the first direct post-transcriptional regulator of erythroid switching, PUM1, that is specifically upregulated in erythroid cells by the master transcription factor, EKLF. Our results indicate that the post-transcriptional regulation by PUM1 may play a crucial role in limiting the production of γ-globin in adult erythroid cells.

While PUM1/2 has been shown to be important for the maintenance of hematopoietic stem cells, their function in erythropoiesis is unknown. We report that among all globins, the ^{A}γ (HBG1) but not the ^{G}γ (HBG2) globin has evolved to harbor a PUM1 binding site in its 3′UTR. This is unlike most other regulators of γ-globin that affect both the duplicated γ-globin genes. Exactly why the ^{A}γ (HBG1) but not the ^{G}γ (HBG2) γ-globin requires this additional layer of regulation is yet unknown. Interestingly the ratio of ^{G}γ (HBG2): ^{A}γ (HBG1) is 3:1 at birth (newborn ratio) and 2:3 in the small amount of HbF present in the blood of adults (adult ratio). How and why this change in the ratio is modulated after the switch to the adult globin takes place, when only about 1% of the fetal form can be detected, is yet to be answered. It would be interesting to test whether this ratio gets skewed in the mutant PUM1 background and understand the relevance of this change, although the ^{G}γ (HBG2): ^{A}γ (HBG1) ratio observed in HPFH has been known to vary. Also, whether and how PUM1 itself is regulated in this developmental window would be an important question to pursue.

We also note the peculiarity of PUM1 that it shares with other regulators of γ-globin such as FOXO3 and BCL11A, in that the mutations in these factors result in neurodegenerative disorders, suggesting a critical role for them in neuronal development. The PUM1 mutation we report here also was in a patient who had PUM1-associated developmental disability, ataxia, and seizure. Why these factors play roles specifically in regulating switching in erythroid cells is unclear. One reason that explains the selection of the switching mechanism from the fetal γ-globin to the adult β form is the enhanced ability of the fetal form to carry oxygen from the placenta to the growing fetus.

β-thalassemia and sickle cell disease, also referred to as β-type hemoglobinopathies, are the most prevalent of the monogenic inherited hemoglobin disorders and present the greatest public health impact in terms of expenditure. Conventional treatment strategies in β-thalassemia and sickle cell anemia range from blood transfusions to bone marrow transplantation; these strategies are invasive and beset with complications such as iron overload over time. Current endeavors towards treatment, including clinical efforts, focus on manipulating key genetic regulators such as enhancers of fetal globin repressors (e.g. BCL11A in erythrocytes), with genome editing. Since PUM1 functions as a cytoplasmic regulator of γ-globin regulation, we propose that it could potentially serve as a safe and effective alternative target towards ameliorating β-thalassemia and sickle cell disease.

### Methods of Treatment

In some aspects, provided herein are methods for the treatment of sickle cell anemia or beta-thalassmia by administering to a patient a composition of an effective amount of an inhibitor of Pumilio-1 (PUM1). The PUM1 inhibitor can be an antibody or fragment thereof, a nucleic acid, or a small molecule. Also disclosed is a pharmaceutical composition comprising an inhibitor of PUM1 and a pharmaceutically acceptable carrier.

Also disclosed herein is a method for increasing fetal hemoglobin levels by administering an effective amount of a composition comprising an inhibitor of Pumilio-1 (PUM1), whereby fetal hemoglobin expression is increased relative to the amount prior to administration of the composition comprising an inhibitor of PUM1. Also disclosed is that the composition can be a small molecule.

In addition, disclosed is a method for increasing fetal hemoglobin levels by conducting genome editing, whereby fetal hemoglobin expression is increased relative to the amount prior to the genome editing. Further disclosed is that the genome editing can decrease PUM1 levels or abolish PUM1 binding to its targets.

In the alternative, also disclosed is a method for increasing fetal hemoglobin levels by utilizing antisense oligonucleotides (such as siRNA), whereby fetal hemoglobin expression is increased relative to the amount prior to the use of the antisense oligonucleotides. Furthermore, the use of antisense oligonucleotides can decrease PUM1.

Also disclosed is a method for increasing fetal hemoglobin level by utilizing RNA decoy technology, whereby fetal hemoglobin expression is increased relative to the amount prior to the use of the RNA decoy technology. The use of RNA decoy technology can decrease PUM1.

Further in accordance with certain aspects of the present invention, the composition suitable for administration is provided in a pharmaceutically acceptable carrier with or without an inert diluent. Except insofar as any conventional media, agent, diluent, or carrier is detrimental to the recipient or to the therapeutic effectiveness of the composition contained therein, its use in administrable composition for use in practicing the therapeutic uses and methods of the present invention is appropriate. Examples of carriers or diluents include fats, oils, water, saline solutions, lipids, liposomes, resins, binders, fillers and the like, or combinations thereof.

In accordance with certain aspects of the present invention, the composition may be combined with the carrier in any convenient and practical manner, i.e., by solution, suspension, emulsification, admixture, encapsulation, absorption, and the like. Such procedures are routine for those skilled in the art.

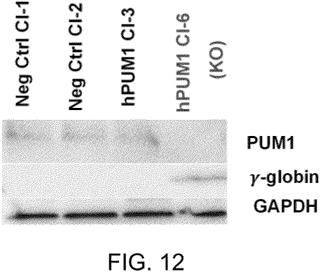

FIG. 12 includes Western blots for PUM1, γ-globin (fetal type beta globin), and loading control GAPDH protein levels are shown. CRISPR-Cas9 based Pumilio1 (PUM1) knockout in human erythroid cells (HUDEP2 cells) recapitulates fetal hemoglobin induction seen upon PUM1 knockdown with shRNAs. Cells were electroporated with Cas9 protein and control sgRNA (Negative control clones- Neg Ctrl Cl-1 and 2) or sgRNA targeting PUM1 (Clone 3 did not show reduction in PUM1 levels, while clone 6 was a knockout for PUM1).

The following article is incorporated by reference herein in its entirety:

• Elagooz R, Dhara AR, Gott RM, Sarah AE, White RA, Ghosh AA, Ganguly S, Man Y, Owusu-Ansa A, Mian OY, Gurkan UA, Komar A, Ramamoorthy M, Gnanapragasam MN. PUM1 mediates the post-transcriptional regulation of human fetal hemoglobin. Blood Advances. 2022 Jun 6; doi: 10.1182/bloodadvances.2021006730. Epub ahead of print. PMID: 35667093

The present disclosure has been described with reference to example embodiments. Modifications and alterations will occur to others upon reading and understanding the preceding detailed description. It is intended that the present disclosure be construed as including all such modifications and alterations insofar as they come within the scope of the appended claims or the equivalents thereof.

1. A method of treating sickle cell anemia or beta-thalassmia, the method comprising:
administering to a patient a composition comprising an effective amount of an inhibitor of Pumilio-1 (PUM1).

2. The method of claim 1, wherein the inhibitor is an antibody or fragment thereof.

3. The method of claim 1, wherein the inhibitor is a nucleic acid.

4. The method of claim 1, wherein the inhibitor is a small molecule.

5. A pharmaceutical composition comprising an inhibitor of a Pumilio-1 and a pharmaceutically acceptable carrier.

6. The composition of claim 5, wherein the inhibitor comprises an antibody or fragment thereof.

7. The composition of claim 5, wherein the inhibitor comprises a nucleic acid.

8. The composition of claim 5, wherein the inhibitor comprises a small molecule.

9. A method for increasing fetal hemoglobin levels, the method comprising:
administering an effective amount of the pharmaceutical composition of claim 5, whereby fetal hemoglobin expression is increased relative to the amount prior to administration of the composition.

10. The method of claim 9, wherein the inhibitor is a small molecule.

11. The method of claim 9, wherein the inhibitor is an antibody or fragment thereof.

12. The method of claim 9, wherein the inhibitor is a nucleic acid.

13. A method for increasing fetal hemoglobin levels, the method comprising:
genome editing, whereby fetal hemoglobin expression is increased relative to the amount prior to the genome editing; and/or
utilizing antisense oligonucleotides such as siRNA, whereby fetal hemoglobin expression is increased relative to the amount prior to the use of antisense oligonucleotides; and/or
utilizing RNA decoy technology, whereby fetal hemoglobin expression is increased relative to the amount prior to the use of the RNA decoy technology.

14. The method of claim 13, wherein the method comprises the genome editing.

15. The method of claim 14, wherein the genome editing decreases PUM1.

16. The method of claim 13, wherein the method comprises the utilizing antisense oligonucleotides such as siRNA.

17. The method of claim 16, wherein the use of antisense oligonucleotides decrease PUM1.

18. The method of claim 13, wherein the method comprises the utilizing RNA decoy technology.

19. The method of claim 18, wherein the use of RNA decoy technology decreases PUM1.

20. The method of claim 13, wherein the fetal hemoglobin level is at least 5% higher in populations treated, than a comparable, control population, wherein no treatment occurs.
